(19) United States
(12) Patent Application Publication
(10) Pub. No.: US 2018/0200263 A1
(43) Pub. Date: Jul. 19, 2018

(54) TOPICAL TREATMENT REGIMENS CONTAINING A PROSTAGLANDIN F2a ANALOG AND A TOPICAL STEROID

(71) Applicant: Pearl E. Grimes, Los Angeles, CA (US)

(72) Inventor: Pearl E. Grimes, Los Angeles, CA (US)

(21) Appl. No.: 15/742,727

(22) PCT Filed: Jul. 6, 2016
(86) PCT No.: PCT/US2016/041065

§ 371 Date: Jan. 8, 2018

Related U.S. Application Data
(60) Provisional application No. 62/189,441, filed on Jul. 7, 2015.

(51) Int. Cl. A61K 31/557; A61K 31/58; A61K 36/82; A61K 38/13; A61K 31/353; A61K 31/436; A61K 9/00; A61P 17/00

(52) U.S. Cl. CPC A61K 31/557; A61K 31/58; A61K 36/82; A61K 38/13; A61K 2300/00; A61K 31/436; A61K 9/0014; A61P 17/00; A61K 31/353

(57) ABSTRACT

Disclosed herein is a topical treatment regimen including a prostaglandin F2α analog and a topical steroid for treating skin conditions, including skin pigmentation disorders associated with hypopigmentation such as vitiligo and nonfacial vitiligo. Examples of prostaglandin F2α analogs include bimatoprost, carboprost, dinoprost, fluprostenol, latanoprost, prostamide F2α, tafluprost, travoprost, and unoprostone, with bimatoprost being preferred. Examples of topical steroids include aclometasone, betamethasone, clobetasol, clobetasone, diflucortolone, fluocinolone, fluprednidene, flurandrenolide, fluticasone, halometasone, hydrocortisone, methylprednisolone, mometasone, prednicarbate, and triamcinolone, with mometasone being preferred. The topical treatment regimen includes one or more topical compositions, with the prostaglandin F2α analog and the topical steroid being contained either in separate topical compositions or in the same topical composition.

### BACKGROUND

Hypopigmentation is the lightening of an area of skin or nails caused by decreased levels of skin pigmentation. There are many diseases, disorders, and conditions associated with hypopigmentation, such as vitiligo, improperly-administered skin-resurfacing treatments, repigmentation therapies such as targeted phototherapy, topical psoralen photochemotherapy, and autologous grafting, as well as skin injuries such as infections, blisters, burns, acne, scrapes, etc.

Melanin is a class of pigment responsible for producing color in parts of the body such as skin. A decrease in production of melanin can result in hypopigmentation. Melanin is produced by melanocytes at the lower layer of the epidermis.

Vitiligo is a condition that causes depigmentation of skin, typically in sections or patches, and affects about 1-2% of the world population. Vitiligo occurs when there is an absence of functional melanocytes in the skin. The precise cause of vitiligo is complex and not yet fully understood. There is some evidence suggesting it is caused by a combination of autoimmune, genetic, and environmental factors. Vitiligo also can affect the mucous membranes and the eye. The average age at vitiligo onset is about 20 years, with onset most commonly observed between the ages of 10 and 30. Approximately 25% of all vitiligo patients are children. The disfiguring aspect of vitiligo can cause psychological trauma, such as stigmatization, avoidance of social situations, anxiousness, and a sense of helplessness.

Vitiligo occurs most often on the face and extremities—typically the hands and wrists. Depigmentation also can occur around the mouth, eyes, nostrils, genitalia, and umbilicus. Depigmented patches are flat areas of normal-feeling skin, and may have a hyperpigmented edge. The edges typically are well-defined but irregular. In trichrome vitiligo, there is an intermediate zone of hypochromia between the achromic center and peripheral unaffected skin.

There are several clinical classifications of vitiligo. Segmental vitiligo presents as one or more macules in a dermatomal or quasidermatomal pattern, and occurs most commonly in children. All other types of vitiligo are classified as non-segmental vitiligo, which is most common. Focal vitiligo is characterized by depigmentation in one area, or macule, such as the trigeminal nerve distribution. Other forms of non-segmental vitiligo often produce symmetric patches, sometimes covering large areas. Mucosal vitiligo affects only mucosal membranes. Generalized vitiligo may be acrofacial, in which depigmentation occurs on the distal fingers and periorificial areas, or vulgaris, which is characterized by widely distributed, scattered patches. Vitiligo universalis manifests as complete or nearly complete depigmentation, and frequently is associated with multiple endocrinopathy syndrome.

### SUMMARY

It has been found that topically administering a prostaglandin F_{2α} analog and a topical steroid can result in a decrease in hypopigmentation when applied to the skin. Examples of prostaglandin F_{2α} analogs include bimatoprost, carboprost, dinoprost, fluprostenol, latanoprost, prostamide F_{2α}, tafluprost, travoprost, and unoprostone. Examples of topical steroids include aclometasone, betamethasone, clobetasol, clobetasone, diflucortolone, fluocinolone, fluprednidene, flurandrenolide, fluticasone, halometasone, hydrocortisone, methylprednisolone, mometasone, prednicarbate, and triamcinolone.

Described herein are topical compositions and topical treatment regimens that include a prostaglandin F_{2α} analog and a topical steroid. The topical treatment regimen can further include calcineurin inhibitors, green tea extracts, or epigallocatechin gallate. Calcineurin inhibitors that can be used in the topical compositions include cyclosporine, tacrolimus, and pimecrolimus. The topical treatment regimens can include topical compositions such as a topical solution, topical cream, topical lotion, topical gel, topical foam, or topical ointment. The topical treatment regimens can be used to treat a subject. The subject can have a skin pigmentation disorder such as hypopigmentation. The topical treatment regimens including a prostaglandin F_{2α} analog and a topical steroid can be used to treat vitiligo or nonfacial vitiligo.

In first aspect, there is a method of treating a skin pigmentation disorder in a subject by topically administering to the subject a prostaglandin F_{2α} analog and a topical steroid.

In example of the first aspect, the skin pigmentation disorder is vitiligo or nonfacial vitiligo.

In another example of the first aspect, the prostaglandin F_{2α} analog is bimatoprost present at an amount between 0.01 and 0.05 wt. %.

In another example of the first aspect, the prostaglandin F_{2α} analog is carboprost, dinoprost, fluprostenol, latanoprost, prostamide F_{2α}, tafluprost, travoprost, or unoprostone present at an amount between 0.01 and 0.05 wt. %.

In another example of the first aspect, the topical steroid is mometasone present at an amount between 0.05 and 0.15 wt. %.

In another example of the first aspect, the topical steroid is aclometasone, betamethasone, clobetasol, clobetasone, diflucortolone, fluocinolone, fluprednidene, flurandrenolide, fluticasone, halometasone, hydrocortisone, methylprednisolone, prednicarbate, or triamcinolone present at an amount between 0.05 and 0.15 wt. %.

In another example of the first aspect, the prostaglandin F_{2α} analog and the topical steroid are present in separate compositions.

In another example of the first aspect, the prostaglandin F_{2α} analog and the topical steroid are present in a single composition.

In a second aspect, there is provided the method of the first aspect, further including administering to the subject cyclosporine, tacrolimus, or pimecrolimus.

In a third aspect, there is provided the method of the first aspect, further including administering to the subject epigallocatechin gallate.

In a fourth aspect, there is provided the method of the first aspect, further including administering to the subject a green tea extract.

In a fifth aspect, there is a method of treating the skin pigmentation disorder nonfacial vitiligo in a subject by topically administering to the subject a topical composition that includes bimatoprost present at amount between 0.01 and 0.05 wt. % and mometasone present at an amount between 0.05 and 0.15 wt. %.

In a sixth aspect, there is a topical composition for treatment of a skin pigmentation disorder, the topical composition including a prostaglandin F_{2α} analog and a topical steroid.

In an example of the sixth aspect, the prostaglandin F_{2α} analog is bimatoprost present at an amount between 0.01 and 0.05 wt. %.

In another example of the sixth aspect, the prostaglandin F_{2α} analog is carboprost, dinoprost, fluprostenol, latanoprost, prostamide F_{2α}, tafluprost, travoprost, or unoprostone present at an amount between 0.01 and 0.05 wt. %.

In another example of the sixth aspect, the topical steroid is mometasone present at an amount between 0.05 and 0.15 wt. %.

In another example of the sixth aspect, the topical steroid is aclometasone, betamethasone, clobetasol, clobetasone, diflucortolone, fluocinolone, fluprednidene, flurandrenolide, fluticasone, halometasone, hydrocortisone, methylprednisolone, prednicarbate, or triamcinolone present at an amount between 0.05 and 0.15 wt. %.

In a seventh aspect, there is provided the topical composition of the sixth aspect, further including cyclosporine, tacrolimus, or pimecrolimus.

In an eighth aspect, there is provided the topical composition of the sixth aspect, further including epigallocatechin gallate.

In a ninth aspect, there is provided the topical composition of the sixth aspect, further including a green tea extract.

In a tenth aspect, there is a method of treating vitiligo in a subject by topically administering once or twice daily to the subject a first topical composition including 0.01 and 0.05 wt. % of a prostaglandin F_{2α} analog (e.g., bimatoprost) and further administering at least once daily a second topical composition including 0.05 and 0.15 wt. % of a topical steroid (e.g., mometasone).

In an example of the tenth aspect, the prostaglandin F_{2α} analog is the sole active ingredient in the first composition.

In another example of the tenth aspect, the topical steroid is sole active ingredient in the second composition.

In another example of the tenth aspect, the first topical composition is administered twice daily and the second topical composition is administered once daily.

In another example of the tenth aspect, the method is for treating nonfacial vitiligo.

In another example of the tenth aspect, the first composition is in the form of a topical solution.

In another example of the tenth aspect, the second composition is in the form of a topical cream.

### DETAILED DESCRIPTION

It has been discovered that a topical treatment regimen including a prostaglandin F_{2α} analog and a topical steroid can be used to treat skin conditions, such as skin pigmentation disorders. Examples of skin pigmentation disorders that the topical treatment regimen can be used to treat include vitiligo or nonfacial vitiligo.

Examples of prostaglandin F_{2α} analogs suitable for the treatment of skin conditions include bimatoprost, carboprost, dinoprost, fluprostenol, latanoprost, prostamide F_{2α}, tafluprost, travoprost, and unoprostone. Herein, references to prostaglandin F_{2α} analogs include salts and derivatives thereof. For example, carboprost is commercially available as the tromethamine salt. Thus, the term carboprost as used herein refers to the free form of carboprost, the tromethamine salt, and any other salts and derivatives of carboprost. Prostaglandin F_{2α} analogs can be used to treatment skin pigmentation disorders such as vitiligo or nonfacial vitiligo. The preferred prostaglandin F_{2α} analog is bimatoprost.

The above examples of prostaglandin F_{2α} analogs have the following structure:

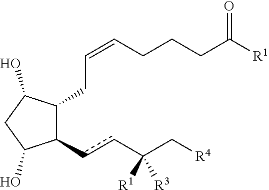

with the following substitutions:

Bimatoprost: R¹ is —NHCH₂CH₃; the bond is a double bond; R² is —H; R³ is —OH; and R⁴ is —CH₂-phenyl.

Carboprost: R¹ is —OH; the bond is a double bond; R² is —CH₃; R³ is —OH; and R⁴ is —CH₂CH₂CH₂CH₃.

Dinoprost: R¹ is —OH; the bond is a double bond; R² is —H; R³ is —OH; and R⁴ is —CH₂CH₂CH₂CH₃.

Fluprostenol: R¹ is —OH; the bond is a double bond; R² is —H; R³ is —OH; and R⁴ is —O-m-trifluoromethylphenyl.

Latanoprost: R¹ is —OCH(CH₃)₂; the bond is a single bond; R² is —H; R³ is —OH; and R⁴ is —CH₂-phenyl.

Prostamide F_{2α}: R¹ is —NHCH₂CH₂OH; the bond is a double bond; R² is —H; R³ is —OH; and R⁴ is —CH₂CH₂CH₂CH₃

Tafluprost: R¹ is —OCH(CH₃)₂; the bond is a double bond; R² is —F; R³ is —F; and R⁴ is —O-phenyl.

Travoprost: R¹ is —OCH(CH₃)₂; the bond is a double bond; R² is —H; R³ is —OH; and R⁴ is —O-m-trifluoromethylphenyl.

Unoprostone: R¹ is —OH; the bond is a single bond; R² and R³ together form ═O; and R⁴ is —CH₂CH₂CH₂CH₂CH₂CH₃.

Examples of topical steroids suitable for the topical compositions and topical treatment regimens of the present disclosure for treatment of skin conditions include aclometasone, betamethasone, clobetasol, clobetasone, diflucortolone, fluocinolone, fluprednidene, flurandrenolide, fluticasone, halometasone, hydrocortisone, methylprednisolone, mometasone, prednicarbate, and triamcinolone. Herein, references to topical steroids include salts and derivatives thereof. For example, aclometasone is commercially available as the dipropionate prodrug. Thus, the term aclometasone as used herein refers the free form of aclometasone, the dipropionate prodrug, and any other salts and derivatives of aclometasone. Topical steroids can be used to treatment skin pigmentation disorders such as vitiligo or nonfacial vitiligo. The preferred topical steroid is mometasone.

The prostaglandin F_{2α} analog and the topical steroid of the topical treatment regimens can be included in separate topical composition or in a single topical composition. These topical compositions can be any type of topical composition, such as a topical solution, a topical cream, a topical lotion, a topical gel, a topical foam, or a topical ointment.

The topical compositions of the topical treatment regimen for treating a skin condition, including the skin pigmentation disorders vitiligo and nonfacial vitiligo, can further include additional ingredients, including additional active agents and excipients. These additional ingredients can be included in a topical composition containing a prostaglandin F_{2α} analog, a topical composition containing a topical steroid, and/or a topical composition containing both a prostaglandin F_{2α} analog and a topical steroid. Additional ingredients can include calcineurin inhibitors. Calcineurin inhibitors that can be used in the topical composition include cyclosporine, tacrolimus, and pimecrolimus, as well as salts or derivatives thereof. Green tea extracts and epigallocatechin gallate also can be included in the topical compositions. Other additional ingredients that can be used in the topical composition include emollients, emulsifiers, solidifiers, surfactants, foamers, thickeners, skin conditioners, absorbents, preservatives, buffering agents, tonicity agents, solvents, complexing agents, diluents, and antioxidants.

The topical treatment regimen can include the simultaneous, sequential, alternating or isolated administration of a prostaglandin F_{2α} analog and a topical steroid. For example, the prostaglandin F_{2α} analog and the topical steroid can be included in a single topical composition and therefore administered simultaneously. The prostaglandin F_{2α} analog and the topical steroid can be included in separate topical compositions that can be mixed together prior to administration and administered simultaneously.

The topical treatment regimen can include once or twice daily administration of the prostaglandin F_{2α} analog and a topical steroid. The daily administration can vary between the prostaglandin F_{2α} analog and topical steroid, for instance, the prostaglandin F_{2α} analog can be administered twice daily in a topical composition and the topical steroid can be administered once daily in a separate topical composition. Another example of a topical treatment regimen can include either first administering the topical composition containing the prostaglandin F_{2α} analog or the topical composition containing the topical steroid followed by the other topical composition within a relatively short period of time (e.g., within 1 to 8 hours). Furthermore, the two topical compositions, each at least containing one of the prostaglandin F_{2α} analog or topical steroid, can be administered in an alternating manner, such as one every morning and the other every evening or one every odd day and the other every even day. Additionally, one of the topical compositions can be administered alone for a period of days and terminated followed by the administration of the other topical composition. Moreover, the topical treatment regimen can include the cyclic on/off periods for either topical composition. For example, the topical composition containing the prostaglandin F_{2α} analog can be administered continuously whereas the topical composition containing the topical steroid can be administered in on/off cycles. Also, a topical composition containing both a prostaglandin F_{2α} analog and a topical steroid can be administered in on/off cycles while a topical composition containing only the prostaglandin F_{2α} can be administered during the off periods of the composition containing both the prostaglandin F_{2α} and the topical steroid.

The topical treatment regimen can include varying doses of the prostaglandin F_{2α} analog and/or the topical steroid. For example, the dosage strength of the prostaglandin F_{2α} analog and/or the topical steroid can be increased or decreased during the treatment period.

The topical treatment regimen disclosed herein can be combined with other known treatment therapies for treating skin conditions, such as the skin pigmentation disorders vitiligo and nonfacial vitiligo.

Herein, when a range such as 5-25 (or 5 to 25) is given, this means preferably at least or more than 5 and, separately and independently, preferably not more than or less than 25. In an example, such a range defines independently at least 5, and separately and independently, not more than 25.

The amount of the prostaglandin F_{2α} analog in the topical compositions disclosed herein can be 0.0005 to 3 wt. %, 0.001 to 2 wt. %, 0.005 to 1 wt. %, 0.01 to 0.5 wt. %, 0.015 to 0.25 wt. %, 0.02 to 0.1 wt. % and 0.03 to 0.05 wt. %. The amount of the topical steroid in the topical compositions disclosed herein can be 0.001 to 5 wt. %, 0.005 to 1 wt. %, 0.01 to 0.5 wt. %, and 0.05 to 0.15 wt. %. If present, the amount of calcineurin inhibitor in the topical compositions disclosed herein can be 0.0005 to 5 wt. %, 0.001 to 1 wt. %, 0.005 to 0.5 wt. %, and 0.01 to 0.2 wt. %. If present, the amount of green tea extract in the topical compositions can include 0.01 to 30 wt. %, 0.02 to 20 wt. %, 0.2 to 10 wt. %. If present, the amount of epigallocatechin gallate in the topical compositions can include 0.005 to 15 wt. %, 0.01 to 10 wt. %, 0.1 to 5 wt. %. If present, the amount of other additional ingredients, for example, emollients, emulsifiers, solidifiers, surfactants, foamers, thickeners, skin conditioners, absorbents, preservatives, buffering agents, tonicity agents, solvents, complexing agents, diluents, and antioxidants can be at conventional weight percent as known in the art. It is preferred that the topical compositions include a dermatologically acceptable carrier useful for topical treatment. Weight percent is disclosed herein as measured based on the total weight of the topical composition for which an individual ingredient is present.

In one or more embodiments, a topical composition can include a prostaglandin F_{2α} analog, a topical steroid or combination of both in the disclosed amounts. For example, a topical composition can include 0.01 to 0.05 wt. % of a prostaglandin F_{2α} analog, 0.05 to 0.15 wt. % of a topical steroid and a dermatologically acceptable carrier.

In another embodiment, a topical composition can include a prostaglandin F_{2α} analog, a topical steroid, a calcineurin inhibitor, a green tea extract, epigallocatechin gallate or a combination thereof in the disclosed amounts. For example, a topical composition can include 0.01 to 0.05 wt. % of a prostaglandin F_{2α} analog, 0.05 to 0.15 wt. % of a topical steroid, and at least one of a calcineurin inhibitor, a green tea extract or epigallocatechin gallate. In another example, a topical composition can include 0.01 to 0.05 wt. % of a prostaglandin F_{2α} analog, 0.05 to 0.15 wt. % of a topical steroid, and at least two of a calcineurin inhibitor, a green tea extract or epigallocatechin gallate.

The following example will serve to illustrate topical treatment of nonfacial vitiligo with a prostaglandin F_{2α} analog and a topical steroid, it being understood that the particulars shown are by way of example and for purpose of illustrative discussion of the preferred embodiments of the present disclosure. The example should not, however, be viewed as limiting the scope of the invention.

### Example

An efficacy and safety study was conducted to compare the effects of bimatoprost monotherapy, mometasone monotherapy, and the combination of bimatoprost and mometasone. 18 randomized subjects completed the 20 week study, five coming in each of the bimatoprost monotherapy and mometasone monotherapy groups with the other eight coming in the bimatoprost/mometasone combination group.

Subjects with vitiligo applied the corresponding drug(s) to depigmented areas on their neck, truck, and extremities. Per FDA request, facial areas where not treated during the study. For the bimatoprost monotherapy group, subjects were instructed to administer bimatoprost twice daily (morning and evening) for all 20 weeks. For the mometasone monotherapy group, subjects were instructed to administer mometasone once daily in the morning for weeks 1-12 and 17-20. Placebo (Cetaphil) was instructed to be administered in the morning for weeks 13-16 and in the evening for all 20 weeks. For the bimatoprost/mometasone combination group, subjects were instructed to administer mometasone once daily in the morning for weeks 1-12 and 17-20 and placebo was administered in the morning for weeks 13-16. Subjects were instructed to administer bimatoprost once daily evening for all 20 weeks. Subjects in either the bimatoprost monotherapy group or the bimatoprost/mometasone combination group the subjects were instructed to administer bimatoprost in the form of a 0.03 wt. % solution which was to be applied in a dose of 1 to 2 drops per 2 cm² body surface area. Subjects in either the mometasone monotherapy group or the bimatoprost/mometasone combination group the subjects were instructed to administer mometasone in the form of 0.1 wt. % mometasone furoate cream which was to be applied in a dose of 1 to 2 pea-sized quantities per anatomic area. The subjects were blind as to which group they were in and therefore which drug(s) they were administering.

During the study period, the subjects were periodically evaluated by an investigator. The investigators evaluated the subjects on a score of 0 to 7 with 0 meaning that the subject's condition was worse, 1 being unchanged, 2 being slight improvement (approximately 10% improvement), 3 being mild improvement (approximately 10 to 25% improvement), 4 being moderate improvement (approximately 25 to 50% improvement), 5 being marked improvement (approximately 50 to 75% improvement), 6 being almost cleared (approximately 75 to 99% improvement), 7 being cleared (100% improvement). Like the subjects, the investigators were blind as to which group the subjects they were evaluating were in and therefore which drug(s) they were evaluating were administering.

Of the five subjects treated with bimatoprost only, two subjects saw moderate improvement (40%) whereas three subjects saw mild improvement (60%) after the treatment regimen was completed. Of the eight subjects treated with bimatoprost and mometasone in combination, five subjects saw moderate improvement (62.5%), two subjects saw mild improvement (25%), and one subject was unchanged (12.5%) after the treatment regimen was completed. Of the five subjects treated with mometasone only, two subjects saw mild improvement (40%) and three subjects saw slight improvement (60%) after the treatment regimen was completed.

While various aspects and embodiments have been disclosed herein, other aspects and embodiments will be apparent to those skilled in the art. The various aspects and embodiments disclosed herein are for purposes of illustration and are not intended to be limiting.

1. A method of treating a skin pigmentation disorder in a subject comprising topically administering to the subject a prostaglandin F_{2α} analog and a topical steroid.

2. The method of claim 1, wherein the skin pigmentation disorder is vitiligo or nonfacial vitiligo.

3. The method of claim 2, wherein the prostaglandin F_{2α} analog is bimatoprost present at an amount between 0.01 and 0.05 wt. %.

4. The method of claim 2, wherein the prostaglandin F_{2α} analog is carboprost, dinoprost, fluprostenol, latanoprost, prostamide F_{2α}, tafluprost, travoprost, or unoprostone present at an amount between 0.01 and 0.05 wt. %.

5. The method of claim 2, wherein the topical steroid is mometasone present at an amount between 0.05 and 0.15 wt. %.

6. The method of claim 1, wherein the topical steroid is aclometasone, betamethasone, clobetasol, clobetasone, diflucortolone, fluocinolone, fluprednidene, flurandrenolide, fluticasone, halometasone, hydrocortisone, methylprednisolone, prednicarbate, or triamcinolone present at an amount between 0.05 and 0.15 wt. %.

7. The method of claim 2, further comprising administering to the subject cyclosporine, tacrolimus, or pimecrolimus.

8. The method of claim 2, further comprising administering to the subject epigallocatechin gallate.

9. The method of claim 2, further comprising administering to the subject a green tea extract.

10. The method of claim 1, wherein the prostaglandin F_{2α} analog and the topical steroid are present in separate compositions.

11. The method of claim 1, wherein the prostaglandin F_{2α} analog and the topical steroid are present in a single composition.

12. A method of treating the skin pigmentation disorder nonfacial vitiligo in a subject comprising topically administering to the subject a topical composition comprising bimatoprost present at amount between 0.01 and 0.05 wt. % and mometasone present at an amount between 0.05 and 0.15 wt. %.

13. A topical composition for treatment of a skin pigmentation disorder, the topical composition comprising a prostaglandin F_{2α} analog and a topical steroid.

14. The topical composition of claim 13, wherein the prostaglandin F_{2α} analog is bimatoprost present at an amount between 0.01 and 0.05 wt. %.

15. The topical composition of claim 13, wherein the prostaglandin F_{2α} analog is carboprost, dinoprost, fluprostenol, latanoprost, prostamide F_{2α}, tafluprost, travoprost, or unoprostone present at an amount between 0.01 and 0.05 wt. %.

16. The topical composition of claim 14, wherein the topical steroid is mometasone present at an amount between 0.05 and 0.15 wt. %.

17. The topical composition of claim 14, wherein the topical steroid is aclometasone, betamethasone, clobetasol, clobetasone, diflucortolone, fluocinolone, fluprednidene, flurandrenolide, fluticasone, halometasone, hydrocortisone, methylprednisolone, prednicarbate, or triamcinolone present at an amount between 0.05 and 0.15 wt. %.

18. The topical composition of claim 14, further comprising cyclosporine, tacrolimus, or pimecrolimus.

19. The topical composition of claim 14, further comprising epigallocatechin gallate.

20. The topical composition of claim 14, further comprising green tea extract.
